(19) United States
(12) Patent Application Publication
(10) Pub. No.: US 2009/0143335 A1
(43) Pub. Date: Jun. 4, 2009

(54) MODIFIED ABSORPTION FORMULATION OF GABOXADOL

(75) Inventors: Mie Larsen, Hvidovre (DK); Carsten Uhd Nielsen, Kobenhavn S (DK); Birger Brodin Larsen, Kobenhavn V (DK); Rene Holm, Jyllinge (DK)

Correspondence Address: DARBY & DARBY P.C., P.O. BOX 770, Church Street Station, New York, NY, 10008-0770, US

(73) Assignee: H. LUNDBECK A/S, Valby-Copenhagen (DK)

(21) Appl. No.: 12/253,879

(22) Filed: Oct. 17, 2008

Related U.S. Application Data
(60) Provisional application No. 60/983,314, filed on Oct. 29, 2007.
(60) Provisional application No. 61/093,524, filed on Sep. 2, 2008.
(60) Provisional application No. 61/105,918, filed on Oct. 16, 2008.

(30) Foreign Application Priority Data
Sep. 1, 2008 (DK) PA 2008 01198

(51) Int. Cl. A61K 31/675; A61K 31/437; A61K 31/43; A61K 31/52; A61K 31/496; C07D 498/02; A61P 25/00; A61K 31/55; A61K 31/5415; A61K 31/545; A61K 31/546

(52) U.S. Cl. 514/81; 514/302; 514/192; 514/209; 514/202; 514/86; 514/263.37; 514/223.5; 514/226.5; 514/254.05; 514/217; 546/116

(57) ABSTRACT

The present invention relates to a pharmaceutical composition comprising gaboxadol or a pharmaceutically acceptable salt thereof and one or more inhibitors of PAT1 and/or one or more inhibitors of OAT. The present invention further relates to a pharmaceutical composition comprising from about 0.5 mg to about 50 mg gaboxadol or a pharmaceutically acceptable salt thereof, wherein the composition provides an in vivo plasma profile comprising a mean Tmax which is longer than about 20 minutes.

### FIELD OF THE INVENTION

The present invention relates to a pharmaceutical composition comprising gaboxadol or a pharmaceutically acceptable salt thereof and one or more inhibitors of PAT1 and/or one or more inhibitors of OAT. The present invention further relates to a pharmaceutical composition comprising from about 0.5 mg to about 50 mg gaboxadol or a pharmaceutically acceptable salt thereof, wherein the composition provides an in vivo plasma profile comprising a mean Tmax which is longer than about 20 minutes.

### BACKGROUND OF THE INVENTION

Gaboxadol (4,5,6,7-tetrahydroisoxazolo [5,4-c]pyridine-3-ol) (THIP) is described in EP Patent No. 0000338 and in EP Patent No. 0840601, and has previously shown great potential in the treatment of sleep disorders and in pre-clinical models of depression (WO2004112786). Gaboxadol has the following general formula:

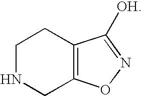

Gaboxadol may be prepared using methods that are well known in the art. For example as disclosed in EP Patent No. 0000338 and in WO2005023820.

WO02094225 discloses a granular preparation containing gaboxadol that can be used for the preparation of solid, shaped pharmaceutical unit dosage forms containing gaboxadol with an immediate release profile.

WO0122941 discloses a melt granulated composition containing gaboxadol and a modified release dosage form prepared from said composition.

In therapeutic dosing with a gaboxadol immediate release formulation, rapid dissolution results in a rapid increase in blood plasma levels of gaboxadol shortly after administration followed by a decrease in blood plasma levels over several hours as gaboxadol is metabolized or eliminated, until sub-therapeutic plasma levels are approached.

Some pharmacological and physiological processes may require a prolonged exposure at therapeutic relevant plasma levels in order to reach optimal therapeutic effects. Thus there is a need for a pharmaceutical dosage form of gaboxadol capable of providing a prolonged exposure at therapeutic relevant plasma levels. Moreover there is a need for a pharmaceutical dosage form of gaboxadol that provides a plasma profile with a later Tmax and/or a decreased Cmax, possibly supplemented with an increase in AUC.

It has now surprisingly been found that it is possible to prepare a formulation of gaboxadol that have demonstrated to alter the absorption of gaboxadol and thereby minimise the peak concentration, extent the Tmax and in special situations further extent the elimination phase of the pharmacokinetic profile (i.e. to increase AUC).

### SUMMARY OF THE INVENTION

In one aspect the present invention relates to a pharmaceutical composition comprising gaboxadol or a pharmaceutically acceptable salt thereof and one or more inhibitors of PAT1 and/or one or more inhibitors of OAT.

In another aspect the present invention relates to a pharmaceutical composition comprising from about 0.5 mg to about 50 mg gaboxadol or a pharmaceutically acceptable salt thereof, wherein the composition provides an in vivo plasma profile comprising a mean Tmax which is longer than about 20 minutes.

### BRIEF DESCRIPTION OF THE DRAWINGS

FIG. 1: Plasma gaboxadol concentrations vs. time profiles after IV dosing in dog. Plasma concentrations of gaboxadol vs. time profiles after an intravenous injection of 2.5 mg/kg gaboxadol (A, (Δ)). Blood samples were collected at 5, 15, 30, 60, 90, 120, 180, 240, 360, 480 and 600 minutes after the drug administration. Shown is mean±S.E.M. of six dogs, n=6. Y-axis: plasma concentration of gaboxadol (ng/ml). X-axis: time (minutes).

FIG. 2: Plasma gaboxadol concentrations vs. time profiles after PO dosing in dog. Plasma gaboxadol concentrations vs. time profiles of beagle dogs after PO administration of 2.5 mg/kg gaboxadol (B, (□)). The same dose was also given with 2.5 mg/kg Trp (C, (▴)), with 10.0 mg/kg Trp (D, (x)), with 50.0 mg/kg Trp (E, (◯)) and with 150.0 mg/kg Trp (F, (▪)). The Trp was given as a co-administration at the same time as gaboxadol. Samples were collected at 5, 15, 30, 60, 90, 120, 180, 240, 360, 480 and 600 minutes after the drug administration. Shown is mean±S.E.M. of six dogs, n=6. Y-axis: plasma concentration of gaboxadol (ng/ml). X-axis: time (minutes).

FIG. 3: Plasma gaboxadol concentrations vs. time profiles after PO dosing in rat. Plasma gaboxadol concentrations vs. time profiles of rats after PO administration of gaboxadol. Dose given was 0.5 mg/kg (G, (◯)) or 5.0 mg/kg (H, ()) of gaboxadol alone or with a pre-incubation of 200.0 mg/kg 5-HTP (I, (□)) or (J, ▪)), respectively. 5-HTP 200.0 mg/kg was given as a pre-incubation 30 min. before gaboxadol. Samples were collected at 5, 15, 30, 45, 60, 120, 240, 360 and 480 minutes after the drug administration. Shown is mean±S.E.M. of five to six rats, n=5-6. Y-axis: plasma concentration of gaboxadol (ng/ml). X-axis: time (minutes).

FIG. 4: Plasma gaboxadol concentrations vs. time profiles after IV dosing in rat. Plasma gaboxadol concentrations vs. time profiles of rats after IV administration of gaboxadol. Rats were given an intravenous injection of 2.5 mg/kg gaboxadol (K, (Δ)). The same dose was also given with 200.0 mg/kg 5-HTP (L, (▴)). 5-HTP 200.0 mg/kg was given as a pre-incubation 30 min. before the gaboxadol. Samples were collected at 5, 15, 30, 45, 60, 120, 240, 360 and 480 minutes after the drug administration. Shown is mean±S.E.M. of five to six rats, n=5-6. Y-axis: plasma concentration of gaboxadol (ng/ml). X-axis: time (minutes).

### DESCRIPTION OF THE INVENTION

The present inventors have found that therapeutic dosing with an immediate release formulation of gaboxadol in some patients with primary insomnia has resulted in dose dependent adverse events. The observed adverse events occurred about the same time as mean Cmax, and disappeared after a few hours after administration, thus the adverse events are correlated to Cmax. The observed adverse events with the immediate release formulation of gaboxadol include dizziness, nausea, vomiting, somnolence, tremor, malaise, sedation, and some psychiatric adverse events. By further analysis of the adverse events, the present inventors found that by reducing the mean Cmax and/or by a longer mean Tmax, the adverse events are rare, milder, and the psychiatric adverse events are non-existing.

The present inventors have found that it is possible to prepare a pharmaceutical composition comprising gaboxadol and one or more inhibitors of PAT1 and/or one or more inhibitors of OAT to provide a modified absorption formulation of gaboxadol. According to the present invention it is possible to modulate the Cmax, the Tmax and in some instances the AUC of gaboxadol by varying the amount of gaboxadol, one or more inhibitors of PAT1 and/or of OAT used in the pharmaceutical composition. The composition according to the present invention gives one or more of the following advantages: a rapid increase in blood plasma levels of gaboxadol can be avoided or diminished, a pharmacokinetic profile of gaboxadol with a later Tmax and/or a decreased Cmax can be achieved, which in some circumstances can be supplemented with an increase in AUC. One or more of the following problems can thus be solved by the present invention: effects associated with a rapid increase in blood plasma levels of gaboxadol can be avoided or diminished while reaching relevant therapeutic blood plasma levels and/or the time interval between dosing with gaboxadol can be extended compared to an immediate release formulation as the therapeutic relevant blood plasma level is maintained over a longer period of time. Thus according to the present invention a pharmaceutical composition comprising gaboxadol is provided, which is capable of reaching therapeutic relevant plasma levels without reaching plasma levels associated with most adverse events, and in some circumstances for an extended period of time.

Thus the present invention relates to a pharmaceutical composition comprising gaboxadol or a pharmaceutically acceptable salt thereof wherein the composition provides a decreased mean Cmax as compared to an immediate release formulation of gaboxadol and still provides therapeutic relevant plasma levels of gaboxadol.

Without being bound by any particular theory it is hypothesized that the PAT1 inhibitor in the composition according to the present invention decreases the absorption rate of gaboxadol from the gastrointestinal tract and thereby provides a modified absorption of gaboxadol. It is further hypothesized that some, maybe all PAT1 inhibitors, and OAT substrates or inhibitors interact with one or more organic anion transporters (OATs) in the kidneys and/or reduce the renal blood flow and thereby also decreases the elimination rate of gaboxadol from the kidneys and thereby provides a blood plasma level of gaboxadol at the therapeutic relevant level over a longer period of time.

The human proton dependent amino acid transporter 1, hPAT1, was cloned from Caco-2 cells in 2003 (Chen, Z. et al. 2003. J Physiol., Vol. 546. Pt 2. 349-361). The transporter belongs to the solute carrier family SLC36 and is the first (SLC36A1) of four. PAT3 and PAT4 are orphan transporters whereas PAT2 is expressed mainly in tissue of lung, heart, kidney, muscle, testis, spleen, adrenal gland, thymus and sciatic nerve. Analyses have discovered hPAT1 mRNA expression ubiquitously in human tissue and it has been detected all along the human gastrointestinal tract with maximal expression in the small intestine, hence making the transporter relevant for absorption of substrates at the hole length of the intestinal tract (Chen, Z. et al. 2003. J Physiol., Vol. 546. Pt 2. 349-361). The amino acid transport via hPAT1 is energized by a significant concentration gradient of protons (H⁺) that is built up across the apical membrane due to an acidic microclimate in the intestinal (Lucas, M. L. et al. 1975. Proc R. Soc Lond. B Biol Sci., Vol. 192. 1106. 39-48).

The Caco-2 cell line can be used as a model of the human small intestinal epithelium. The proton dependent amino acid transporter has previously been characterized thoroughly in this vitro model and also to some extends, in transfected cell systems (Boll, M. et al. 2002. J Biol Chem., Vol. 277. 25. 22966-22973; Chen, Z. et al. 2003. J Physiol., Vol. 546. Pt 2. 349-361). By competition assays as well as translocation experiments, various compounds have been tested for interaction with PAT1. According to these in vitro characterizations of a PAT1 substrate refers to a compound that is transported across a (21-28 days old) Caco-2 cell monolayer with a flux increasing with the transmembrane pH gradient. Furthermore, by addition of a high concentration of another PAT1 substrate, which could be L-Proline but not limited to, this transport must be inhibited.

A PAT1 inhibitor refers to a compound that decreases the transport of PAT1 substrates across a Caco-2 cell monolayer. The inhibitor can act in a competitive or non-competitive manner, depending if it binds the transporter in the substrate pocket or not.

Classic PAT1 substrates are small zwitterionic unbranched α-amino acids like glycine, alanine, serine and proline in addition to some β-amino acids as β-alanine and AIB (α-(Methylamino)-isobutyric acid) as well as a few γ-amino acids like GABA (γ-amino butyric acid) (Metzner, L. et al. 2006. Amino. Acids., Vol. 31. 2. 111-117). Some xenobiotics have been demonstrated to be among the hPAT1 substrates, e.g. the neuromodulatory and antibacterial agent D-cycloserine. Also several GABA receptor blockers and reuptake inhibitors as well as proline analogues used in treatment of cancer and fibrotic diseases are transported by PAT1 (Metzner, L. et al. 2006. Amino. Acids., Vol. 31. 2. 111-117).

Possible competitive PAT1 inhibitors includes but are not limited to: Glycine, L-Alanine, D-Alanine, L-Serine, D-Serine, L-Proline, D-Proline, GABA (γ-amino butyric acid), Sarcosine, Betaine, N-Methyl-L-alanine (AIB (α-(Methylamino)-isobutyric acid)), D-cycloserine, β-Alanine, Vigabatrine, Guvacine, TACA (trans-4-aminocrotonic acid).

Possible PAT1 inhibitors could be but is not limited to: 5-hydroxy-tryptophan (5-HTP), Serotonin (5-HT), L-tryptophan (Trp), Tryptamine, Indole-3-propionic acid.

The organic anion transporters (OATs) were identified in 1997. The transporter belongs to the SLC22 gene family (Koepsell, H. et al. 2004. Pflugers. Arch., Vol. 447. 5. 666-676) and are characterised by a remarkable broad substrate specificity. The currently known transporters include OAT1-4 and URAT1, which are mainly located in kidneys (Rizwan, A. N. et al. 2007. Pharm. Res., Vol. 24. 3. 450-470), hence several publications have focused on the transporters contribution to renal secretion of xenobiotics and drugs (for review see Burckhardt, B. C. et al. 2003. Rev Physiol Biochem Pharmacol., Vol. 146. 95-158). Expression have also been reported in the brain, especially in the choroids plexus and the blood brain barrier (Pritchard, J. B. et al. 1999. J Biol Chem., Vol. 274. 47. 33382-33387), the eyes, the skeletal muscle and several organs in different stages of embryo development (Pavlova, A. et al. 2000. Am. J Physiol Renal Physiol., Vol. 278. 4. F635-F643). OATs do not directly utilize ATP hydrolysis for energtisation of substrate translocation. Most, if not all members of the OAT family operate as anion exchangers, i.e. they couple the uptake of an organic anion into the cell to the release of another organic anion from the cell. Thereby, OAT utilize existing intracellular>extracellular gradients of anions, e.g. α-ketoglutarate, lactate and nicotinate, to drive uphill uptake of organic anions against the negative membrane potential. In the kidney proximal tubule, OAT are functionally couples to Na⁺-driven mono- and dicarboxylate transporters that establish and maintain the intracellular>extracellular gradients of lactate, nicotinate and α-ketoglutarate (Rizwan, A. N. et al. 2007. Pharm. Res., Vol. 24. 3. 450-470).

Typical substrates of OATs have a molecular weight of up to 400-500 (Sekine, T. et al. 2006. Am. J Physiol Renal Physiol., Vol. 290. 2. F251-F261; Wright, S. H. et al. 2004. Am. J Physiol Renal Physiol., Vol. 287. 3. F442-F451), and the specificity are very broad in terms of chemical structures being transported by the OATs, including but not limited to: Kynurenate, Xanthurenate, 5-hydroxyindol acetate, p-aminohippurate, 6-carboxyflurescein, Benzylpenicillin, Cefadroxil, Cefamadole, Cefazolin, Cefoperazone, Cefotamime, Cephalexine, Cephalotin, Cephradine, Acylovir, Adefovir, Cidofovir, Ganciclovir, Tenofovir, Valacylovir, Zidovudine, Acetazolamide, Bumetanide, Chlorothiazide, Ethacrynate, Furosemide, Hydrochlorothiazide, Methazolamide, Trichloromethiazide, Acetaminophen, Acetylsalicylate Dilofenac, Diflusinal, Etodolac, Flurbiprofen, Ibuprofen, Indomethacin, Ketoprofen, Loxoprofen, Mefanamate, Naproxen, Phenacetin, Piroxicam, Salicylate, Sulidac. An OAT substrate is here defined by a compound, which is transported into oocytes transfected with OAT mRNA, with a significant increased rate compared to a control situation.

### DEFINITIONS

Cmax is defined as the highest plasma drug concentration estimated during an experiment (ng*ml¹). Tmax is defined as the time when Cmax is estimated (min). AUC is the total area under the plasma drug concentration-time curve, from drug administration until the drug is eliminated (ng*min*ml⁻¹). The area under the curve is governed by clearance. Clearance is defined as the volume of blood or plasma that is totally cleared of its content of drug per unit time (ml*hr⁻¹*kg⁻²). Elimination rate constant relates to the amount of drug in the body, which is eliminated per unit time is defined as the velocity with which the drug is eliminated (hr⁻¹) (Gabrielsson and Weiner. 2007. Pharmacokinetic and Pharmacodynamic Data Analysis, Concepts and Applications, 4th ed., CRC Press, Baco Raton, Fla. ISBN 978-9-1976-5100-4).

The term “PK” refers to the pharmacokinetic profile.

As used herein, the term “subject” refers to any warm-blooded species such as human and animal. The subject, such as a human, to be treated with gaboxadol may in fact be any subject of the human population, male or female, which may be divided into children, adults, or elderly. Any one of these patient groups relates to an embodiment of the invention.

As used herein, the term “treating” or “treatment” refers to preventing or delaying the appearance of clinical symptoms of a disease or condition in a subject that may be afflicted with or predisposed to the disease or condition, but does not yet experience or display clinical or subclinical symptoms of the disease or condition. “Treating” or “treatment” also refers to inhibiting the disease or condition, i.e., arresting or reducing its development or at least one clinical or subclinical symptom thereof. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician. Nonetherless, prophylactic (preventive) and therapeutic (curative) treatment are two separate embodiments of the invention.

As used herein, the term “pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

According to a first aspect the present invention relates to a pharmaceutical composition comprising gaboxadol or a pharmaceutically acceptable salt thereof and one or more inhibitors of PAT1 and/or one or more inhibitors of OAT. In one embodiment of the first aspect of the invention the composition comprises one or more inhibitors of PAT1 but not an inhibitor of OAT. In another embodiment of the first aspect of the invention the composition comprises one or more inhibitors of OAT but not an inhibitor of PAT1. In another embodiment of the first aspect of the invention the composition comprises both one or more inhibitors of PAT1 and one or more inhibitors of OAT. In another embodiment of the first aspect of the invention gaboxadol is in the form of an acid addition salt, or a zwitter ion hydrate or zwitter ion anhydrate. In another embodiment of the first aspect of the invention gaboxadol is in the form of a pharmaceutically acceptable acid addition salt selected from the hydrochloride or hydrobromide salt, or in the form of the zwitter ion monohydrate. In another embodiment of the first aspect of the invention the amount of gaboxadol ranges from 0.5 mg to 50 mg. In another embodiment of the first aspect of the invention the composition is an oral dose form. In another embodiment of the first aspect of the invention the composition is a solid oral dose form, such as tablets or capsules, or a liquid oral dose form. In another embodiment of the first aspect of the invention said gaboxadol is crystalline. In another embodiment of the first aspect of the invention PAT1 is human PAT1. In another embodiment of the first aspect of the invention the inhibitor of PAT1 is selected from 5-hydroxy-tryptophan (5-HTP), L-Proline, D-Proline, Sarcosine, L-Alanine, D-Alanine, N-Methyl-L-alanine, N-Methyl-D-alanine, α-(Methylamino)-isobutyric acid, Betaine, D-cycloserine, L-cycloserine, β-Alanine, Serotonin, L-tryptophan, D-tryptophan, Tryptamine, Indole-3-propionic acid. In another embodiment of the first aspect of the invention the amount of PAT1 inhibitor ranges from about 0.5 to about 3000 mg, such as about 1, 5, 10, 25, 50, 100, 150, 200, 250, 300, 350, 400, 450, 500, 750, 1000, 1250, 1500, 1750, 2000, 2250, 2500, 2750 or 3000 mg. In another embodiment of the first aspect of the invention OAT is human OAT. In another embodiment of the first aspect of the invention the inhibitor of OAT is selected from Kynurenate, Xanthurenate, 5-hydroxyindol acetate, p-aminohippurate, 6-carboxyflurescein, Benzylpenicillin, Cefadroxil, Cefamadole, Cefazolin, Cefoperazone, Cefotamime, Cephalexine, Cephalotin, Cephradine, Acylovir, Adefovir, Cidofovir, Ganciclovir, Tenofovir, Valacylovir, Zidovudine, Acetazolamide, Bumetanide, Chlorothiazide, Ethacrynate, Furosemide, Hydrochlorothiazide, Methazolamide, Trichloromethiazide, Acetaminophen, Acetylsalicylate Dilofenac, Diflusinal, Etodolac, Flurbiprofen, Ibuprofen, Indomethacin, Ketoprofen, Loxoprofen, Mefanamate, Naproxen, Phenacetin, Piroxicam, Salicylate, Sulidac. In another embodiment of the first aspect of the invention the amount of OAT inhibitor ranges from about 0.5 to about 500 mg, such as about 1, 5, 10, 25, 50, 100, 150, 200, 250, 300, 350, 400, 450 or 500 mg. In another embodiment of the first aspect of the invention the composition comprises one or more excipients. In another embodiment of the first aspect of the invention the composition comprises a compound, which is a serotonin reuptake inhibitor, or any other compound which causes an elevation in the level of extracellular serotonin. In another embodiment of the first aspect of the invention the serotonin uptake inhibitor is selected from citalopram, escitalopram, fluoxetine, sertraline, paroxetine, fluvoxamine, venlafaxine, duloxetine, dapoxetine, nefazodone, imipramin, femoxetine and clomipramine or a pharmaceutically acceptable salt of any of these compounds. In another embodiment of the first aspect of the invention the serotonin uptake inhibitor is escitalopram, as the base or a pharmaceutically acceptable salt thereof, such as the oxalate, hydrobromide or hydrochloride salt.

According to a second aspect the present invention relates to a pharmaceutical composition comprising from about 0.5 mg to about 50 mg gaboxadol or a pharmaceutically acceptable salt thereof, wherein the composition provides an in vivo plasma profile comprising a mean Tmax which is longer than about 20 minutes. In one embodiment of the second aspect of the invention said mean Tmax is longer than about 25, 30, 35, 40, 45, 50, 55, 60, 65, 70 or 75 minutes. In another embodiment of the second aspect of the invention the composition provides an in vivo plasma profile comprising a mean Cmax of less than about 2250 ng/ml. In another embodiment of the second aspect of the invention said mean Cmax is less than about 2000, 1750, 1500, 1250, 1000, 750, 500, 250, 200 or 100 ng/ml. In another embodiment of the second aspect of the invention the composition provides an in vivo plasma profile comprising a mean AUC_{0-∞} of more than about 8.000 ng·min·ml⁻¹. In another embodiment of the second aspect of the invention said mean AUC_{0-∞} is more than about 16.000, 20.000, 40.000, 80.000, 120.000 or 200.000 ng·min·ml⁻¹. In another embodiment of the second aspect of the invention the clearance is lower than 40 ml/min. In another embodiment of the second aspect of the invention said clearance is lower than 30 ml/min, 20 ml/min, 10 ml/min or 5 ml/min. In another embodiment of the second aspect of the invention the composition comprises about 2 mg gaboxadol or a pharmaceutically acceptable salt thereof and provides an in vivo plasma profile comprising: a mean Tmax of more than about 20 minutes; a mean Cmax of less than about 100 ng/ml; and a mean AUC_{0-∞} of more than about 8.000 ng·min·ml⁻¹. In another embodiment of the second aspect of the invention the composition comprises about 4 mg gaboxadol or a pharmaceutically acceptable salt thereof and provides an in vivo plasma profile comprising: a mean Tmax of more than about 20 minutes; a mean Cmax of less than about 200 ng/ml; and a mean AUC_{0-∞} of more than about 16.000 ng·min·ml⁻¹. In another embodiment of the second aspect of the invention the composition comprises about 5 mg gaboxadol or a pharmaceutically acceptable salt thereof and provides an in vivo plasma profile comprising: a mean Tmax of more than about 20 minutes hours; a mean Cmax of less than about 250 ng/ml; and a mean AUC_{0-∞} of more than about 20.000 ng·min·ml⁻¹. In another embodiment of the second aspect of the invention the composition comprises about 10 mg gaboxadol or a pharmaceutically acceptable salt thereof and provides an in vivo plasma profile comprising: a mean Tmax of more than about 20 minutes; a mean Cmax of less than about 500 ng/ml; and a mean AUC_{0-∞} of more than about 40.000 ng·min·ml⁻¹. In another embodiment of the second aspect of the invention the composition comprises about 20 mg gaboxadol or a pharmaceutically acceptable salt thereof and provides an in vivo plasma profile comprising: a mean Tmax of more than about 20 minutes; a mean Cmax of less than about 1000 ng/ml; and a mean AUC_{0-∞} of more than about 80.000 ng·min·ml⁻¹. In another embodiment of the second aspect of the invention the composition comprises about 30 mg gaboxadol or a pharmaceutically acceptable salt thereof and provides an in vivo plasma profile comprising: a mean Tmax of more than about 20 minutes; a mean Cmax of less than about 1500 ng/ml; and a mean AUC_{0-∞} of more than about 120.000 ng·min·ml⁻¹. In another embodiment of the second aspect of the invention the composition comprises about 50 mg gaboxadol or a pharmaceutically acceptable salt thereof and provides an in vivo plasma profile comprising: a mean Tmax of more than about 20 minutes; a mean Cmax of less than about 2500 ng/ml; and a mean AUC_{0-∞} of more than about 200.000 ng·min·ml⁻¹. In another embodiment of the second aspect of the invention the clearance is lower than 40 ml/min and the AUC higher than 200.000 ng·min·ml⁻¹. In another embodiment of the second aspect of the invention said mean Tmax, Cmax and/or AUC_{0-∞} is obtained when the composition is administered to a dog and said clearance is obtained when the composition is administered to a dog or rat.

In another embodiment of the second aspect of the invention said mean Tmax is longer than about 30 minutes. In another embodiment of the second aspect of the invention the composition provides an in vivo plasma profile comprising a mean Cmax of less than about 300 ng/ml. In another embodiment of the second aspect of the invention the amount of gaboxadol is selected from about 2.5 mg, about 5 mg or about 10 mg. In another embodiment of the second aspect of the invention the amount of gaboxadol is 2.5 mg, mean Cmax is less than about 40 ng/ml, such as 35 about ng/ml, 30 ng/ml, 25 ng/ml or 20 ng/ml, and mean Tmax is longer than about 1 hour, such as 1.5 hours, 2 hours or 2.5 hours. In another embodiment of the second aspect of the invention the amount of gaboxadol is 5 mg, mean Cmax is less than about 85 ng/ml, such as 80 about ng/ml, 75 ng/ml, 70 ng/ml or 65 ng/ml, and mean Tmax is longer than about 1 hour, such as 1.5 hours, 2 hours or 2.5 hours. In another embodiment of the second aspect of the invention the amount of gaboxadol is 10 mg, mean Cmax is less than about 150 ng/ml, such as 145 about ng/ml, 140 ng/ml, 135 ng/ml or 130 ng/ml, and mean Tmax is longer than about 1 hour, such as 1.5 hours, 2 hours or 2.5 hours. In another embodiment of the second aspect of the invention said mean Tmax and Cmax is obtained when the composition is administered to a human. In another embodiment of the second aspect of the invention gaboxadol is in the form of an acid addition salt, or a zwitter ion hydrate or zwitter ion anhydrate. In another embodiment of the second aspect of the invention gaboxadol is in the form of a pharmaceutically acceptable acid addition salt selected from the hydrochloride or hydrobromide salt, or in the form of the zwitter ion monohydrate. In another embodiment of the second aspect of the invention the composition is an oral dose form. In another embodiment of the second aspect of the invention the composition is a solid oral dose form, such as tablets or capsules, or a liquid oral dose form. In another embodiment of the second aspect of the invention said gaboxadol is crystalline. In another embodiment of the second aspect of the invention the composition comprises one or more excipients.

In one embodiment of the invention, the pharmaceutical composition provides a mean Cmax corresponding to 80% such as 75%, 70%, or 65% of the Cmax observed with an immediate release formulation of gaboxadol. Furthermore the present invention relates to a pharmaceutical composition comprising gaboxadol or a pharmaceutically acceptable salt thereof wherein the composition provides a mean Tmax which is longer than is observed with an immediate release formulation of gaboxadol and still provides therapeutic relevant plasma levels of gaboxadol.

In a further embodiment a compound provides inhibition of both PAT1 and OAT.

In a further embodiment, wherein the mean Tmax, Cmax and/or AUC_{0-∞} is obtained when the composition of the invention is administered to a dog, said dog is a beagle and said beagle is fasted 20-24 hours (h) before administration of said composition.

In a further embodiment, wherein the clearance is obtained when the composition of the invention is administered to a dog, said dog is a beagle and said beagle is fasted 20-24 hours (h) before administration of said composition.

In a further embodiment, wherein the clearance is obtained when the composition of the invention is administered to a rat, said rat is a male Sprague-Dawley rat (Charles River Laboratories, Wilmington, Mass., USA) and said rat is maintained on standard food and water until 16-20 hours prior to administration of said composition.

In a further embodiment, the pharmaceutical composition of the present invention is for the treatment of a sleep disorder, such as primary insomnia, or depression, such as major depression.

Throughout this description, “gaboxadol” is intended to include any form of the compound, such as the free base (zwitter ion), pharmaceutically acceptable salts, e.g., pharmaceutically acceptable acid addition salts, hydrates or solvates of the base or salt, as well as anhydrates, and also amorphous, or crystalline forms.

In a further embodiment, gaboxadol is selected from the zwitter ion, typically a hydrate thereof, although the anhydrate is also suitable. A suitable embodiment is the zwitter ion monohydrate.

In a further embodiment, gaboxadol is selected from an acid addition salt, typically a pharmaceutically acceptable acid addition salt. A suitable embodiment is an organic acid addition salt, such as any one of the maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. Another suitable embodiment is an inorganic acid addition salt, such as any one of the hydrochloric, hydrobromic, sulfuric, sulfamic, phosphoric or nitric acid addition salts.

In another embodiment, gaboxadol is in the form of the hydrochloric acid salt, the hydrobromic acid salt, or the zwitter ion monohydrate.

In a further embodiment, gaboxadol is crystalline, such as the crystalline hydrochloric acid salt, the crystalline hydrobromic acid salt, or the crystalline zwitter ion monohydrate.

In a further embodiment, the pharmaceutical composition of the present invention does contain hydrophilic cellulose ether polymer, such as hydroxypropylmethylcellulose, such as Metolose 90SH-15.000 and Metolose 90SH-100.000.

The acid addition salts according to the invention may be obtained by treatment of gaboxadol with the acid in an inert solvent followed by precipitation, isolation and optionally re-crystallization by known methods and if desired micronization of the crystalline product by wet or dry milling or another convenient process, or preparation of particles from a solvent-emulsification process. Suitable methods are described in EP Patent No. 0000338, for example.

Precipitation of the salt of gaboxadol is typically carried out in an inert solvent, e.g., an inert polar solvent such as an alcohol (e.g., ethanol, 2-propanol and n-propanol), but water or mixtures of water and inert solvent may also be used.

Gaboxadol may be administered as an oral dose form, such as a solid oral dose form, typically tablets or capsules, or as a liquid oral dose form. Gaboxadol may be administered in an immediate release dosage form or a controlled or sustained release dosage form. According to one embodiment, the dosage form provides controlled or sustained release of the gaboxadol in an amount less than a sleep-inducing amount. Gaboxadol may be conveniently administered orally in unit dosage forms, such as tablets or capsules, containing the active ingredient in an amount from about 0.1 to about 150 mg/day, from about 0.2 to about 100 mg/day, from about 0.5 to about 50 mg/day, from about 0.1 to about 50 mg/day, from about 1 to about 15 mg/day, or from about 2 to about 5 mg/day. Typically, the pharmaceutical composition comprises from about 0.5 mg to about 20 mg, such as about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg or about 20 mg of gaboxadol. The amount of gaboxadol is calculated based on the free base (zwitter ion) form.

In one embodiment, gaboxadol is administered once daily (for example, in the morning or afternoon) using doses of about 2.5 mg to about 20 mg. In another embodiment gaboxadol is administered twice daily.

According to the present invention, gaboxadol or a pharmaceutically acceptable salt thereof may be administered in any suitable way, e.g., orally or parenterally, and it may be presented in any suitable form for such administration, e.g., in the form of tablets, capsules, powders, syrups or solutions or dispersions for injection. In another embodiment, and in accordance with the purpose of the present invention, gaboxadol is administered in the form of a solid pharmaceutical entity, suitably as a tablet or a capsule or in the form of a suspension, solution or dispersion for injection. Additionally, gaboxadol may be administered with a pharmaceutically acceptable carrier, such as an adjuvant and/or diluent.

The invention also relates to a pharmaceutical composition or kit comprising gaboxadol and a compound, which is a serotonin reuptake inhibitor (SRI), or any other compound which causes an elevation in extracellular 5-HT, and optionally pharmaceutically acceptable carriers or diluents.

In an embodiment, the SRIs is selected from citalopram, escitalopram, fluoxetine, sertraline, paroxetine, fluvoxamine, duloxetine, venlafaxine, duloxetine, dapoxetine, nefazodone, imipramin, femoxetine and clomipramine. Just to clarify, each of these SRIs constitute individual embodiments, and may be the subject of individual claims.

The term selective serotonin reuptake inhibitor (SSRI) means an inhibitor of the monoamine transporters which has stronger inhibitory effect at the serotonin transporter than the dopamine and the noradrenaline transporters.

Selective serotonin reuptake inhibitors (SSRIs) are among the most preferred serotonin reuptake inhibitors used according to the present invention. Thus, in a further embodiment the SRI is selected from SSRIs, such as citalopram, escitalopram, fluoxetine, fluvoxamine, sertraline or paroxetine.

Citalopram is preferably used in the form of the hydrobromide or as the base, escitalopram in the form of the oxalate, fluoxetine, sertraline and paroxetine in the form of the hydrochloride and fluvoxamine in the form of the maleate.

Serotonin reuptake inhibitors, including the SSRIs specifically mentioned hereinabove, differ both in molecular weight and in activity. As a consequence, the amount of serotonin reuptake inhibitor used in combination therapy depends on the nature of said serotonin reuptake inhibitor. In one embodiment of the invention, the serotonin reuptake inhibitor or the compound causing an increase in the level of extracellular 5-HT, is administered at lower doses than required when the compound is used alone. In another embodiment, the serotonin reuptake inhibitor or the compound causing an increase in the level of extracellular 5-HT, is administered in normal doses.

In a further embodiment the pharmaceutical composition comprising gaboxadol and a compound, which is a serotonin reuptake inhibitor (SRI), or any other compound which causes an elevation in extracellular 5-HT, and optionally pharmaceutically acceptable carriers or diluents may be administered as an oral dose form, such as a solid oral dose form, typically tablets or capsules, or as a liquid oral dose form. The composition may be administered in an immediate release dosage form or in a controlled or sustained release dosage form.

Methods for the preparation of solid or liquid pharmaceutical preparations are well known in the art. See, e.g., Remington: The Science and Practice of Pharmacy, 21st ed., Lippincott Williams & Wilkins (2005). Tablets may thus be prepared by mixing the active ingredients with excipients known in the art, such as an ordinary carrier, such as an adjuvant and/or diluent, and subsequently compressing the mixture in a tabletting machine. Non-limiting examples of adjuvants and/or diluents include: corn starch, lactose, mannitol calcium phosphate, microcrystalline cellulose, talcum, magnesium stearate, gelatine, gums, and the like. Any other adjuvant or additive such as colourings, aroma, and preservatives may also be used provided that they are compatible with the active ingredients.

All non-patent references, patents, and patent applications cited and discussed in this specification are incorporated herein by reference in their entirety and to the same extent as if each was individually incorporated by reference.

### REFERENCE LIST

1. Boll, M. et al. (2004) Pflugers. Arch. Vol. 447. 5. 776-779
2. Boll, M. et al. (2002) J Biol Chem. Vol. 277. 25. 22966-22973
3. Burckhardt, B. C. et al. (2003) Rev Physiol Biochem Pharmacol. Vol. 146. 95-158
4. Chen, Z. et al. (2003) J Physiol. Vol. 546. Pt 2. 349-361
5. Gabrielsson, J. and Weiner, D. (2007) Pharmacokinetic and Pharmacodynamic Data Analysis, Concepts and Applications, 4th ed., CRC Press, Baco Raton, Fla. ISBN 978-9-1976-5100-4
6. Koepsell, H. et al. (2004) Pflugers. Arch. Vol. 447. 5. 666-676
7. Lucas, M. L. et al. (1975) Proc R. Soc Lond. B Biol Sci. Vol. 192. 1106. 39-48
8. Metzner, L. et al. (2006) Amino. Acids. Vol. 31. 2. 111-117
9. Pavlova, A. et al. (2000) Am. J Physiol Renal Physiol. Vol. 278. 4. F635-F643
10. Pritchard, J. B. et al. (1999) J Biol Chem. Vol. 274. 47. 33382-33387
11. Rizwan, A. N. et al. (2007) Pharm. Res. Vol. 24. 3. 450-470
12. Sekine, T. et al. (2006) Am. J Physiol Renal Physiol. Vol. 290. 2. F251-F261
13. Wright, S. H. et al. (2004) Am. J Physiol Renal Physiol. Vol. 287. 3. F442-F451

### EXAMPLES

### Example 1

This example describes data from a study conducted in beagle dogs.

### Materials

4,5,6,7-tetrahydroisoxazolo[5,4-c]pyridin-3-ol hydrochloride/C₆H₈N₂O₂, HCl, (gaboxadol hydrochloride) and the deuto substituted form C₆H₄D₄N₂O₂, HCL, (deuto-gaboxadol hydrochloride) were supplied by H. Lundbeck. 5-Hydroxy-L-tryptophan (5-HTP), L-tryptophan (Trp), L-proline (Pro), acetonitrile (ACN) and methanol were obtained from Sigma-Aldrich (St. Louis, Mo., USA). Acetic acid was from MERCK. Heparin, 5000 IE/a.e./ml was purchased from LEO (Ballerup, Dk).

### Methods

### In Vivo Study

Prior to commencement of the studies the protocols were approved by the Animal Welfare Committee, appointed by the Danish Ministry of Justice and all animal procedures were carried out in compliance with EC Directive 86/609/EEC, the Danish law regulating experiments on animals and NIH Guidelines for the Care and Use of Laboratory Animals. 6 full-grown male beagle dogs (body weight 15.9-21.7 kg) were selected and allocated into a roman quadrant design and assigned to receive all 6 formulations of gaboxadol hydrochloride randomly during 6 weeks. The dogs were fasted 20-24 hours before the initiation of the experiment and fed again 10 hours after the administration. The gaboxadol dose was given either as an intravenous injection (1.0 ml/kg) or as an oral solution given by gavage (5.0 ml/kg) directly into the stomach using a soft tube. All dogs received 2.5 mg/kg gaboxadol. In addition to gaboxadol, the oral formulations contained 0, 2.5, 10.0, 50.0 or 150.0 mg/kg of tryptophan to ensure simultaneous co-administration of the two compounds. All solutions were adjusted to a pH of 5.2 and osmolarity was checked with a Vapro vapour pressure osmometer (model 5520, Wescor Inc. Logan, Utah, USA), the intravenous solutions were adjusted to isoosmolarity with glucose. Blood samples of 2.0 ml were taken from the vena cephalica by individual vein puncture and collected into Eppendorf tubes containing 200 IE heparin as anticoagulant. Samples were collected before administration of gaboxadol and after 5, 15, 30, 60, 90 minutes and 2, 3, 4, 6, 8 and 10 hours after gaboxadol administration. The plasma was harvested immediately by centrifugation for 15 minutes at 2200 g and 4-8° C. and stored at −80° C. until further analysis. After each day of gaboxadol dosing the animals had 6 days of washout.

The pharmacokinetics (PK) was evaluated in WinNonlin. Plasma concentrations curves of the animals dosed intravenously were fitted to a 2-compartment model whereas the data from animals dosed orally were analysed in a non-compartment model. Statistical analysis was done in Sigma Stat.

### Quantitative Analysis by HPLC and MS/MS Detection.

Gaboxadol was extracted from plasma and HBSS⁺ samples by liquid extraction. 100 μl HBSS⁺ (80 μl purified water were added to the 20 μl samples) or 100 μl plasma samples were mixed with 25 μl intern standard (d₄-gaboxadol) and 25 μl purified water. Protein precipitation was carried out by addition of 400 μl cold acetonitrile. After centrifugation at 10,000 g in 15 minutes, 425 μl supernatant was transferred to glass tubes and evaporated to dryness under nitrogen at 45° C. The samples were re-solved in 80 μl methanol/acetonitrile (30:70), whirl mixed for 10 minutes and centrifuged in 3 minutes at 3000 rpm, before transferal to medium well plates and placed at 10° C. in the autosampler. Gaboxadol concentration in the extracted samples was subsequently quantified by hydrophilic interaction chromatography (HILIC-chromatography) followed by MS/MS detection. The LC system comprised of an Agilent 1100 series pump and degasser, a CTC Analytics interface transferred data to the computer and a Peltier Thermostat and HTC Pal autosampler handled the samples. An Asahipak amino column, (NH₂P-50, 150×2 mm) from Phenomenex was used for the chromatographic separation with a mobile phase of 20.0 mM ammoniumacetat pH=4:acetonitrile (30:70) and a flow rate of 0.2 ml/min. 20 μL samples were injected onto the column, which was kept at room temperature. The total runtime was 10 minutes with the first 5 minutes of elution let to waste. The elution time of gaboxadol on the column was approximately 8 minutes. The MS/MS system used consisted of a Sciex API 4000 MS/MS detector with a Turbo Ion Spray and Turbo V source (Applied Biosystems). The detection was performed in negative ionization mode where gaboxadol (precursor 139.1 Da, product 110.1 Da) and d₄-gaboxadol (precursor 143.0, product 112.2 Da) were measured by multiple-reaction-monitoring (MRM). The signals were linear between 0.5 and 2500.0 ng/ml and the limit of quantification by this procedure was 0.5 ng/ml. The software was from Analyst™ (Applied Biosystem, version 4.0).

### Results and Discussion

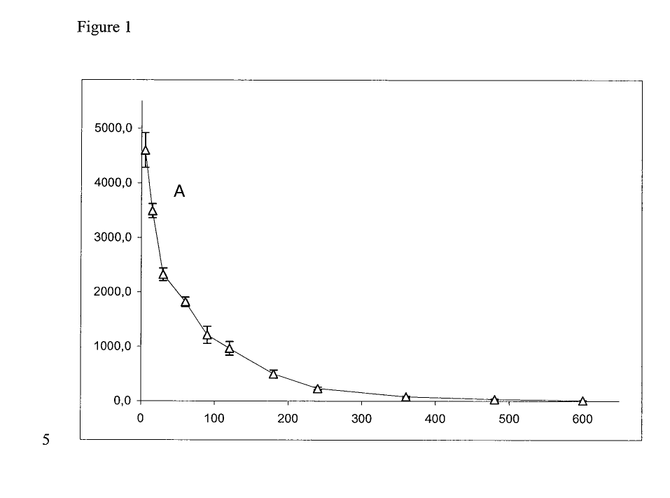

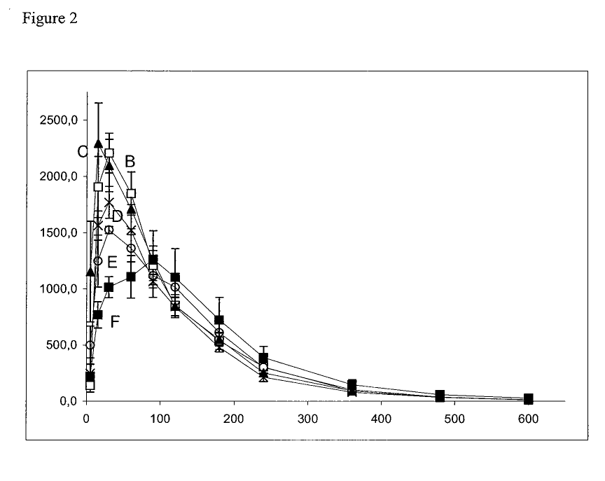

The plasma concentrations versus time profiles are presented in FIGS. 1 and 2.

TABLE 1
Pharmacokinetic parameters obtained from the animals in example 1.

| | Group | | | | | |
|---|---|---|---|---|---|---|
| | A (IV) | B | C | D | E | F |
| Tryptophan | 0 | 0 | 2.5 | 10 | 50 | 150 |
| (mg/kg) | | | | | | |
| kₑ (hr⁻¹) | 1.02 ± 0.14 | 0.46 ± 0.02 | 0.50 ± 0.03 | 0.44 ± 0.03 | 0.50 ± 0.03 | 0.48 ± 0.07 |
| AUC | 5618 ± 377 | 4715 ± 248 | 4760 ± 350 | 4032 ± 339 | 4294 ± 211 | 4405 ± 801 |
| (hr · ng · ml⁻¹) | | | | | | |
| Tₘₐₓ (hr) | — | 0.46 ± 0.12 | 0.35 ± 0.13 | 0.54 ± 0.15 | 0.46 ± 0.12 | 1.50 ± 0.39 |
| | | | | | | ** |
| Cₘₐₓ (ng/ml) | 5489 ± 404 | 2502 ± 43 | 2473 ± 178 | 1868 ± 114 | 1662 ± 37 | 1419 ± 161 |
| | | | | ** | *** | *** |
| CL (ml/hr/kg) | 456 ± 32 | 538 ± 29 | 537 ± 34 | 645 ± 59 | 589 ± 26 | 700 ± 152 |
| Fₐ | — | 85.3 ± 5.7 | 86.1 ± 6.7 | 75.0 ± 10.4 | 78.2 ± 6.3 | 79.7 ± 14.5 |

** Significant statistical difference from formulation B, P < 0.01 in a Pairwise Multiple Tukey comparison test.
*** Significant statistical difference from formulation B, P < 0.005 in a Pairwise Multiple Tukey comparison test.
IV (A) and PO (B-F) administration of 2.5 mg gaboxadol/kg. Data represents mean ± SEM, n = 6.

The bioavailability, Fₐ, of gaboxadol after oral administration in dog was found to be 85.3±5.7% (Table 1). Oral coadministration of 2.5-150 mg/kg tryptophan did not change the AUC of gaboxadol significantly, and the mean relative bioavailability of the formulations varied between 75.0% (10 mg/kg tryptophan) and 86.1% (2.5 mg/kg tryptophan). Likewise, the elimination rate constants (kₑ) and the clearance (CL) of gaboxadol did not change by coadministration of tryptophan. However, tryptophan coadministration decreased the maximal gaboxadol plasma concentration, Cₘₐₓ, by 57% from 2502 ng/ml to 1419 ng/ml in the absence and presence of 150 mg/kg tryptophan (p<0.001). Furthermore, the time required to reach the maximal plasma concentration, Tₘₐₓ, was increased from 0.46 hour to 1.5 hours (p<0.01). The changes in the Cₘₐₓ-values of the five dose groups clearly indicated a direct interaction between gaboxadol and tryptophan

Based upon these data it is evident that Trp has an effect on the absorption profile of gaboxadol. This effect is considered to be mediated by the two compounds interacting with the PAT1 transporter, i.e. in situations of high Trp doses, gaboxadol can not be transported by the PAT1, as many of the binding sites are taken up by Trp. Co-administration of a compound that inhibits or is a substrate to the PAT1 may consequently modify the absorption profile of gaboxadol.

### Example 2

This example describes data from a study conducted in rats.

### Materials

As in example 1

### Experimental Methods

As in example 1, with the following exception:

### Oral Formulations

0.05 or 0.5 mg gaboxadol as well as 0.0 or 20.0 mg 5-HTP was dissolved in purified water pr ml. at room temperature and placed on ice in ultrasound for 10 min. The formulations were adjusted to pH 4-5 and with NaOH/HCl and made isotonic by addition of mannitol. pH of all solutions was adjusted to pH above 4.0 and below 5.0, the osmolality was adjusted with mannitol to 280 mmol/kg.

### In Vivo Experiments

Male Sprague-Dawley rats (Charles River Laboratories, Wilmington, Mass., USA) of 220-240 gram were housed and acclimated for 7 days before entering the experiments. The rats were maintained on standard food and water until 16-20 hours prior to dosing when food was retrieved to insure complete gastric emptying before experiments were conducted. Water was available to the animals until beginning of experiment and again 2 hours after. Each animal was randomly assigned to receive either one of the intravenous or oral formulations.

6 parallel groups of rats (n=6) were given isotonic solutions of 0.5 or 5.0 mg/kg of gaboxadol together with saline or 200.0 mg/kg 5-HTP, by oral gavage (10.0 ml/kg). The suspensions of 5-HTP were given as a pre-incubation 30 min. prior to the gaboxadol solutions.

Blood samples of 0.2 ml were taken from the tail vein by individual vein puncture and collected into plasma collection tubes containing 20 IE heparin. Samples were collected at 5, 15, 30, 45, 60 minutes and after 2, 3, 4, 6, 8 hours after gaboxadol administration. The plasma was harvested immediately by centrifugation for 10 min. at 3.600 g and stored at −80° C. until further analysis.

At the conclusion of the experiment the animals were euthanized.

### Results and Discussion

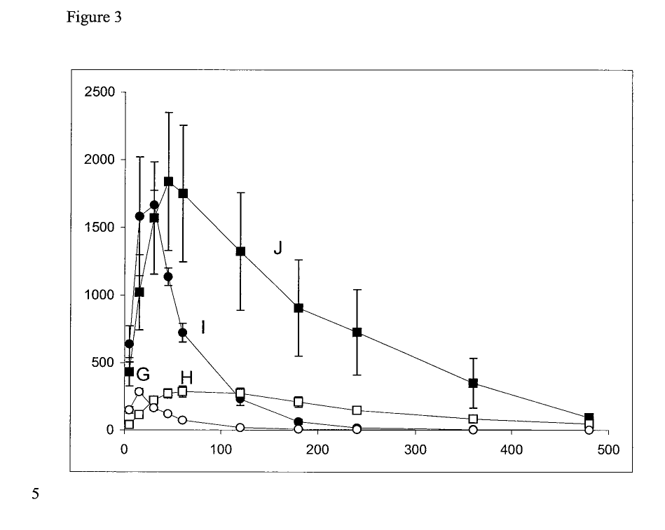

The plasma concentrations versus time profiles are presented in FIG. 3.

TABLE 2
Pharmacokinetic parameters obtained from the animals in example
2, PO administration of 0.5 and 5.0 mg gaboxadol/kg.

| | Treatment | | | |
|---|---|---|---|---|
| | G | H | I | J |
| Gaboxadol (mg/kg) | 0.5 | 0.5 | 5.0 | 5.0 |
| 5-HTP (mg/kg) | — | 200.0 | — | 200.0 |
| Kₑ ± S.E.M. (min⁻¹) | 0.0164 ± 0.0021 | 0.0038 ± 0.0004 | 0.0199 ± 0.0017 | 0.0058 ± 0.0008 |
| T_{1/2} (min) | 46 | 190 | 36 | 141 |
| AUC ± S.E.M. | 13980 ± 1273 | 75403 ± 12665 | 114055 ± 10058 | 379724 ± 126717 |
| (ng · min · ml⁻¹) | | | | |
| Tₘₐₓ ± S.E.M. (min) | 16 ± 3.3 | 63 ± 12.5 | 20 ± 3.2 | 43 ± 2.5 |
| Cₘₐₓ ± S.E.M. (ng/ml) | 273 ± 26.7 | 294 ± 39.4 | 2061 ± 153.1 | 1854 ± 509.4 |

Data represents mean ± SEM, n = 6.

As seen in FIG. 3 and Table 2 absorption of gaboxadol after oral administration happened mostly within 15-20 minutes after dosing as the plasma concentration of gaboxadol increased in this period. After 15-20 minutes elimination of gaboxadol was increasing and the plasma concentration of gaboxadol decreased. The time of peak plasma gaboxadol concentration (Tₘₐₓ) was postponed from 16 to 63 minutes when 200 mg 5-HTP/kg was given as a pre-incubation before 0.5 mg gaboxadol/kg. When rats were given 5.0 mg gaboxadol/kg the Tₘₐₓ was postponed from 20 to 43 minutes after pre-incubation with 5-HTP. The maximum plasma concentration Cₘₐₓ did not seem to change by pre-incubation of 5-HTP.

When the animals were dosed with gaboxadol and PAT1 inhibitor 5-HTP, the AUC increased compared to control animals (not dosed with 5-HTP). The dose of 5-HTP (200 mg/kg) was 40 or 400 times higher than the dose of gaboxadol (5.0 or 0.5 mg/kg) and the AUC increased by 330 and 540% compared to the control groups. The AUC may be increased because of a decreased elimination rate. The gaboxadol elimination rate constant was reduced to about 25% when 5-HTP was present.

Taken together, the absorption of gaboxadol seems to be altered by co-administration of the PAT inhibitor 5HTP. Further the elimination of gaboxadol seems affected by interaction with the PAT, OAT or other transporters which 5-HTP interacts with.

### Example 3

### Materials

As in example 1

### Experimental Methods

As in example 2, with the following exception:

### Intravenous Formulations

0.25 mg gaboxadol as well as 0.0 or 10.0 mg 5-HTP was dissolved in purified water pr ml. at room temperature and placed on ice in ultrasound for 10 min. The solutions of gaboxadol used for intravenous injection was filtered through a 0.45 μm filter.

Animals were administered with 100.0 mg/kg 5-HTP or saline by oral gavage 30 min. prior to intravenous injection of 2.5 mg/kg gaboxadol into the tail vein (5.0 ml/kg).

### Results and Discussion

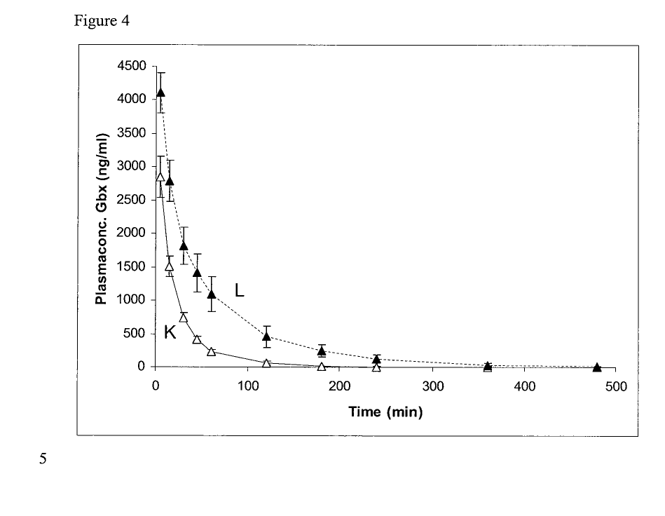

The plasma concentrations versus time profiles are presented in FIG. 4.

TABLE 3
Pharmacokinetic parameters obtained from the animals in
example 3, IV administration of 2.5 mg gaboxadol/kg.

| | Treatment | |
|---|---|---|
| | K | L |
| Gaboxadol (mg/kg) | 2.5 | 2.5 |
| 5-HTP (mg/kg) | — | 100 |
| Kₑ ± S.E.M. (min⁻¹) | 0.0266 ± 0.0008 | 0.0181 ± 0.0030 |
| T_{1/2} (min) | 26 | 44 |
| AUC ± S.E.M. (ng · min · ml⁻¹) | 72546 ± 6145 | 213756 ± 44021 |
| Tₘₐₓ ± S.E.M.(min) | 5 ± 0.0 | 5 ± 0.0 |
| Cₘₐₓ ± S.E.M. (ng/ml) | 2854 ± 312.2 | 4109 ± 302.6 |

Data represents mean ± SEM, n = 6.

The plasma profile of the IV group pre-incubated with 5-HTP (group L) was different from that of rats in the group that received only gaboxadol (group K). The AUC of group K was almost 3 times as big as group L, which probably was caused by a smaller elimination rate constant Kₑ (Table 3). These results suggest that 5-HTP interfere with the clearance of gaboxadol by interaction with the OAT or other transporters.

AUC-dose-linearity was observed for gaboxadol as the AUC of group G (0.5 mg gaboxadol/kg) was five times the size of group K (2.5 mg gaboxadol/kg) and AUC of group J (5 mg/kg) is almost 10 times the size of group G.

1. A pharmaceutical composition comprising gaboxadol or a pharmaceutically acceptable salt thereof and one or more inhibitors of PAT1 and/or one or more inhibitors of OAT.

2. The composition of claim 1 comprising one or more inhibitors of PAT1 but not an inhibitor of OAT.

3. The composition of claim 1 comprising one or more inhibitors of OAT but not an inhibitor of PAT1.

4. The composition of claim 1 comprising both one or more inhibitors of PAT1 and one or more inhibitors of OAT.

5. The composition of claim 1 wherein gaboxadol is in the form of an acid addition salt, or a zwitter ion hydrate or zwitter ion anhydrate.

6. The composition of claim 1 wherein gaboxadol is in the form of a pharmaceutically acceptable acid addition salt selected from the hydrochloride or hydrobromide salt, or in the form of the zwitter ion monohydrate.

7. The composition of claim 1 wherein the amount of gaboxadol ranges from 0.5 mg to 50 mg.

8. The composition of claim 1 wherein the composition is an oral dose form.

9. The composition of claim 1 wherein the composition is a solid oral dose form, such as tablets or capsules, or a liquid oral dose form.

10. The composition of claim 1 wherein said gaboxadol is crystalline.

11. The composition of claim 1 wherein PAT1 is human PAT1.

12. The composition of claim 1 wherein the inhibitor of PAT1 is selected from 5-hydroxy-tryptophan (5-HTP), L-Proline, D-Proline, Sarcosine, L-Alanine, D-Alanine, N-Methyl-L-alanine, N-Methyl-D-alanine, α-(Methylamino)-isobutyric acid, Betaine, D-cycloserine, L-cycloserine, β-Alanine, Serotonin, L-tryptophan, D-tryptophan, Tryptamine, Indole-3-propionic acid.

13. The composition of claim 1 wherein the amount of PAT1 inhibitor ranges from about 0.5 to about 3000 mg.

14. The composition of claim 1 wherein OAT is human OAT.

15. The composition of claim 1 wherein the inhibitor of OAT is selected from Kynurenate, Xanthurenate, 5-hydroxyindol acetate, p-aminohippurate, 6-carboxyflurescein, Benzylpenicillin, Cefadroxil, Cefamadole, Cefazolin, Cefoperazone, Cefotamime, Cephalexine, Cephalotin, Cephradine, Acylovir, Adefovir, Cidofovir, Ganciclovir, Tenofovir, Valacylovir, Zidovudine, Acetazolamide, Bumetanide, Chlorothiazide, Ethacrynate, Furosemide, Hydrochlorothiazide, Methazolamide, Trichloromethiazide, Acetaminophen, Acetylsalicylate Dilofenac, Diflusinal, Etodolac, Flurbiprofen, Ibuprofen, Indomethacin, Ketoprofen, Loxoprofen, Mefanamate, Naproxen, Phenacetin, Piroxicam, Salicylate, Sulidac.

16. The composition of claim 1 wherein the amount of OAT inhibitor ranges from about 0.5 to about 500 mg, such as about 1, 5, 10, 25, 50, 100, 150, 200, 250, 300, 350, 400, 450 or 500 mg.

17. The composition of claim 1 comprising one or more excipients.

18. The composition of claim 1 comprising a compound, which is a serotonin reuptake inhibitor, or any other compound which causes an elevation in the level of extracellular serotonin.

19. The composition of claim 18 wherein the serotonin uptake inhibitor is selected from citalopram, escitalopram, fluoxetine, sertraline, paroxetine, fluvoxamine, venlafaxine, duloxetine, dapoxetine, nefazodone, imipramin, femoxetine and clomipramine or a pharmaceutically acceptable salt of any of these compounds.

20. The composition of claim 18 wherein the serotonin uptake inhibitor is escitalopram, as the base or a pharmaceutically acceptable salt thereof, such as the oxalate, hydrobromide or hydrochloride salt.

21. A pharmaceutical composition comprising from about 0.5 mg to about 50 mg gaboxadol or a pharmaceutically acceptable salt thereof, wherein the composition provides an in vivo plasma profile comprising a mean Tmax which is longer than about 20 minutes.

22. The composition of claim 21 wherein said mean Tmax is longer than about 25 minutes.

23. The composition of claim 21, wherein the composition provides an in vivo plasma profile comprising a mean Cmax of less than about 2250 ng/ml.

24. The composition of claim 23, wherein said mean Cmax is less than about 2000 ng/ml.

25. The composition of claim 21, wherein the composition provides an in vivo plasma profile comprising a mean AUC_{0-∞} of more than about 8.000 ng·min·ml⁻¹.

26. The composition of claim 25, wherein said mean AUC_{0-∞} is more than about 16.000 ng·min·ml⁻¹.

27. The composition of claim 21, where the clearance is lower than 40 ml/min.

28. The composition of claim 27 wherein said clearance is lower than 30 ml/min.

29. The composition of claim 21, wherein the composition comprises about 2 mg gaboxadol or a pharmaceutically acceptable salt thereof and provides an in vivo plasma profile comprising: a mean Tmax of more than about 20 minutes; a mean Cmax of less than about 100 ng/ml; and a mean AUC_{0-∞} of more than about 8.000 ng·min·ml⁻¹.

30. The composition of claim 21, wherein the composition comprises about 4 mg gaboxadol or a pharmaceutically acceptable salt thereof and provides an in vivo plasma profile comprising: a mean Tmax of more than about 20 minutes; a mean Cmax of less than about 200 ng/ml; and a mean AUC_{0-∞} of more than about 16.000 ng·min·ml⁻¹.

31. The composition of claim 21, wherein the composition comprises about 5 mg gaboxadol or a pharmaceutically acceptable salt thereof and provides an in vivo plasma profile comprising: a mean Tmax of more than about 20 minutes hours; a mean Cmax of less than about 250 ng/ml; and a mean AUC_{0-∞} of more than about 20.000 ng·min·ml⁻¹.

32. The composition of claim 21, wherein the composition comprises about 10 mg gaboxadol or a pharmaceutically acceptable salt thereof and provides an in vivo plasma profile comprising: a mean Tmax of more than about 20 minutes; a mean Cmax of less than about 500 ng/ml; and a mean AUC_{0-∞} of more than about 40.000 ng·min·ml⁻¹.

33. The composition of claim 21, wherein the composition comprises about 20 mg gaboxadol or a pharmaceutically acceptable salt thereof and provides an in vivo plasma profile comprising: a mean Tmax of more than about 20 minutes; a mean Cmax of less than about 1000 ng/ml; and a mean AUC_{0-∞} of more than about 80.000 ng·min·ml⁻¹.

34. The composition of claim 21, wherein the composition comprises about 30 mg gaboxadol or a pharmaceutically acceptable salt thereof and provides an in vivo plasma profile comprising: a mean Tmax of more than about 20 minutes; a mean Cmax of less than about 1500 ng/ml; and a mean AUC_{0-∞} of more than about 120.000 ng·min·ml⁻¹.

35. The composition of claim 21, wherein the composition comprises about 50 mg gaboxadol or a pharmaceutically acceptable salt thereof and provides an in vivo plasma profile comprising: a mean Tmax of more than about 20 minutes; a mean Cmax of less than about 2500 ng/ml; and a mean AUC_{0-∞} of more than about 200.000 ng·min·ml⁻¹.

36. The composition of claim 21, where the clearance is lower than 40 ml/min and the AUC higher than 200.000 ng·min·ml⁻¹.

37. The composition of claim 21 wherein said mean Tmax, Cmax and/or AUC_{0-∞} is obtained when the composition is administered to a dog and said clearance is obtained when the composition is administered to a dog or rat.

38. The composition of claim 21, wherein said mean Tmax is longer than about 30 minutes.

39. The composition of claim 21, wherein the composition provides an in vivo plasma profile comprising a mean Cmax of less than about 300 ng/ml.

40. The composition of claim 21, wherein the amount of gaboxadol is selected from about 2.5 mg, about 5 mg or about 10 mg.

41. The composition of claim 21, wherein the amount of gaboxadol is 2.5 mg, mean Cmax is less than about 40 ng/ml, and mean Tmax is longer than about 1 hour.

42. The composition of claim 21, wherein the amount of gaboxadol is 5 mg, mean Cmax is less than about 85 ng/ml, and mean Tmax is longer than about 1 hour.

43. The composition of claim 21, wherein the amount of gaboxadol is 10 mg, mean Cmax is less than about 150 ng/ml, and mean Tmax is longer than about 1 hour.

44. The composition of claim 38, wherein said mean Tmax and Cmax is obtained when the composition is administered to a human.

45. The composition of claim 21 wherein gaboxadol is in the form of an acid addition salt, or a zwitter ion hydrate or zwitter ion anhydrate.

46. The composition of claim 21 wherein gaboxadol is in the form of a pharmaceutically acceptable acid addition salt selected from the hydrochloride or hydrobromide salt, or in the form of the zwitter ion monohydrate.

47. The composition of claim 21 wherein the composition is an oral dose form.

48. The composition of claim 21 wherein the composition is a solid oral dose form, such as tablets or capsules, or a liquid oral dose form.

49. The composition of claim 21 wherein said gaboxadol is crystalline.

50. The composition of claim 21 comprising one or more excipients.

51. The composition of claim 21 wherein said mean Tmax is longer than about 75 minutes.

52. The composition of claim 23, wherein said mean Cmax is less than about 100 ng/ml.

53. The composition of claim 25, wherein said mean AUC_{0-∞} is more than about 200.000 ng·min·ml⁻¹.

54. The composition of claim 27 wherein said clearance is lower than 5 ml/min.
